(19) United States
(12) Patent Application Publication
(10) Pub. No.: US 2024/0050635 A1
(43) Pub. Date: Feb. 15, 2024

(54) HOLLOW FIBER MASS TRANSFER DRIVE FOR EXTRACORPOREAL BLOOD CIRCULATION

(71) Applicant: CHINABRIDGE (SHENZHEN) MEDICAL TECHNOLOGY CO., LTD., Shenzhen, Guangdong (CN)

(72) Inventor: Andrea Zaniboni, San Martino Spino (IT)

(21) Appl. No.: 18/260,770

(22) PCT Filed: Dec. 8, 2021
(86) PCT No.: PCT/CN2021/136347

§ 371 Date: Jul. 7, 2023

Related U.S. Application Data
(60) Provisional application No. 63/264,545, filed on Nov. 24, 2021.

(51) Int. Cl. A61M 1/16; B01D 69/08; B01D 63/04

(52) U.S. Cl. CPC A61M 1/1698; A61M 1/1625; A61M 1/1629; B01D 69/08; B01D 63/043; A61M 2202/0014; A61M 2202/0028; A61M 2202/0007; A61M 2202/0208; A61M 2202/0225; Y10S 261/28

(57) ABSTRACT

A device for extracorporeal blood circulation includes a potted body having an oxygenator module including at least two of a plurality of circular hollow fiber mat layers, a heat exchanger module including at least two of the plurality of circular hollow fiber mat layers, a blood inlet port adjacent a first end of the potted body and a blood outlet port adjacent a second end of the potted body. The device includes a gas inlet and outlet port and a fluid inlet and outlet port. A first of the plurality of hollow fiber mats includes a plurality of hollow fibers aligned in a direction that is angled relative to a direction of alignment of the plurality of hollow fibers of a second of the plurality of hollow fiber mats and, the plurality of hollow fiber mats are aligned to have the same orientation as one another.

### TECHNICAL FIELD

The disclosure relates to the field of devices for extracorporeal circulation of blood. More specifically, the disclosure relates to extracorporeal blood oxygenators and components thereof, such as an oxygenator and heat exchanger.

### BACKGROUND

Devices for blood extracorporeal circulation may include dual portions of an oxygenator and heat exchanger to exchange O₂ and CO₂ between blood and a gas mixture and exchange heat between blood and a heating or cooling fluid through the walls of semipermeable hollow fiber membranes. Blood contacts the outside surfaces of the hollow fibers while the gas mixture and the heating/cooling fluid (a water solution) are circulated inside the hollow fiber lumens. In the devices using this technology, the hollow fibers may be organized in different ways. They may be in a single or multifilament form which is woven around a core or may be structured in mats wound around a core or stacked in mat layers on top of one another (without a core). Various examples of this technology are well known in the technical field.

There remains a need for a blood extracorporeal circulation device wherein there are reduced high blood side pressure drops which may be damaging to blood cells, as well as a device that reduces the clot formation in the blood, while accomplishing a simple formation and manufacturing process.

### SUMMARY

Embodiments of the present invention include an oxygenation hollow fiber membrane organized in stacked hollow fiber mat layers with a structure expressly conceived to provide higher-gas exchange efficiency and better oxygenation effect, a preparation method thereof, and an oxygenation assembly made using the hollow fiber membrane that is easier to be manufactured.

According to one example (“Example 1”), an oxygenation device for use in connection with extracorporeal blood circulation includes a potted body including a first alignment feature defining an alignment axis, an oxygenator module including a plurality of circular hollow fiber mat layers including a second alignment feature configured to align with the first alignment feature of the potted body, such that each of the plurality of hollow fiber mat layers is oriented with respect to the alignment axis, a heat exchanger module including at least two of the plurality of circular hollow fiber mat layers, a blood inlet port positioned adjacent a first end of the potted body, and a blood outlet port positioned adjacent a second end of the potted body that is opposite the first end. The device further includes a gas inlet port and a gas outlet port configured for guiding a gas mixture through a plurality of hollow fibers of the plurality of hollow fiber mat layers, and a fluid inlet port and a fluid outlet port configured for providing and removing heater/cooler (H/C) fluid through a portion of the potted body. The device further includes wherein a first layer of the plurality of hollow fiber mat layers includes a first plurality of parallel hollow fibers disposed at a first angle relative to the alignment axis and a second layer of the plurality of hollow fiber mat layers includes a second plurality of parallel hollow fibers disposed at a second angle relative to the alignment axis, the first and second angles disposed on opposite sides of the alignment axis.

According to a second example, (“Example 2”), the device of Example 1 includes wherein the gas mixture is configured to pass through the at least two of the plurality of hollow fiber mat layers of the oxygenator module.

According to a third example, (“Example 3”), the device of Example 1 includes wherein the H/C fluid is configured to pass through the at least two of the plurality of hollow fiber mat layers of the heat exchanger module.

According to a fourth example, (“Example 4”), the device of Example 1 includes wherein the potted body further comprises a blood distribution grid positioned between the blood inlet port and the heat exchanger module.

According to a fifth example, (“Example 5”), the device of Example 1 includes wherein the potted body further comprises a blood separation grid positioned between the heat exchanger module and the oxygenator module.

According to a sixth example, (“Example 6”), the device of Example 1 includes wherein the potted body further comprises a blood collection grid positioned between the oxygenator module and the blood outlet port.

According to a seventh example, (“Example 7”), the device of Example 1 includes a first housing portion placed adjacent the first end of the potted body and a second housing portion positioned adjacent the second end of the potted body.

According to an eighth example, (“Example 8”), the device of Example 1 includes wherein the plurality of hollow fiber mat layers each include a plurality of threads coupled to the plurality of hollow fibers.

According to a ninth example, (“Example 9”), the device of Example 8 wherein the plurality of threads are aligned at an angle that is not orthogonal to the alignment of the plurality of hollow fibers.

According to a tenth example, (“Example 10”), an assembly of a plurality of hollow fiber mat layers for use in an extracorporeal blood circulation device includes a first hollow fiber mat layer and a second hollow fiber mat layer, positioned directly adjacent one another, each of the first and second hollow fiber mat layers includes a plurality of parallel threads and a plurality of parallel hollow fibers. The assembly further includes wherein the plurality of threads of the first and second hollow fiber mats are positioned parallel to an alignment axis, and wherein the plurality of hollow fibers of the first hollow fiber mat layer are parallel to a second axis disposed at a first angle with respect to the alignment axis and the plurality of hollow fibers of the second hollow fiber mat layer are parallel to a third axis disposed at a second angle with respect to the alignment axis and on an opposite side of the alignment axis as the first angle.

According to an eleventh example, (“Example 11”), the assembly of Example 10 includes wherein the first and second hollow fiber mat layers are circular in shape.

According to a twelfth example, (“Example 12”), the assembly of Example 10 includes wherein the second axis is angled relative to the third axis by between 0 and 50 degrees.

According to a thirteenth example, (“Example 13”), the assembly of Example 12 includes wherein each of the plurality of hollow fiber mat layers comprises at least two grooves that align with the at least two grooves of the hollow fiber mat layers positioned adjacent one another.

According to a fourteenth example, (“Example 14”), the assembly of Example 13 includes wherein each of the plurality of hollow fiber mat layers are aligned to have the same orientation as one another.

According to a fifteenth example, (“Example 15”), the assembly of Example 12 further includes wherein the plurality of hollow fiber mat layers comprise a third hollow fiber mat layer positioned directly below the second hollow fiber mat layer, and a fourth hollow fiber mat layer positioned directly below the third hollow fiber mat layer.

According to a sixteenth example, (“Example 16”), the assembly of Example 15 further includes wherein a plurality of hollow fibers of the third hollow fiber mat layer are aligned along the second axis and a plurality of hollow fibers of the fourth hollow fiber mat layer are aligned with the third axis.

According to a seventeenth example, (“Example 17”), an oxygenation device for use in connection with extracorporeal blood circulation includes a potted body including a blood inlet disposed near a first end and a blood outlet disposed near a second end opposing the first end, and a lateral external surface including a first alignment feature defining an alignment axis. The oxygenator module includes a plurality of circular hollow fiber mat layers including a plurality of hollow fibers coupled to a plurality of threads, and each of the layers including a second alignment feature configured to align with the first alignment feature of the potted body, such that each of the plurality of hollow fiber mat layers is oriented with respect to the alignment axis. The device further includes a gas inlet port configured to provide a gas mixture to the plurality of hollow fibers and wherein a first layer of the plurality of hollow fiber mat layers comprises a first plurality of parallel hollow fibers disposed at a first angle relative to the alignment axis and a second layer of the plurality of hollow fiber mat layers, disposed adjacent to the first layer, includes a second plurality of parallel hollow fibers disposed at a second angle relative to the alignment axis, the first and second angles disposed on opposite sides of the alignment axis.

According to an eighteenth example, (“Example 18”), the device of Example 17 further includes wherein the first and second angles are opposite angles with respect to the alignment axis.

According to a nineteenth example, (“Example 19”), the device of Example 17 further includes wherein each of the first and second angles is between 0 and 25 degrees.

According to a twentieth example, (“Example 20”), the device of Example 19 further includes wherein in each of the hollow fiber mat layers, the plurality of threads are disposed at an angle that is not orthogonal to the alignment of the plurality of hollow fibers.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

The accompanying drawings are included to provide a further understanding of the disclosure and are incorporated in and constitute a part of this specification, illustrate an embodiment, and together with the description serve to explain the principles of the disclosure.

FIG. 1 is a schematic of a patient undergoing extracorporeal blood circulation,

FIG. 2 is a schematic front view of an oxygenator device, according to some embodiments of the present disclosure,

FIG. 3 is a lateral isometric view from the left of the oxygenator device of FIG. 2, according to embodiments of the present disclosure,

FIG. 4 is a lateral cross section of the oxygenator device taken along line A-A of FIG. 2,

FIG. 5 is an exploded view of the oxygenator device of FIG. 2, according to embodiments of the present disclosure,

FIG. 6 is a perspective view of a potted body of the oxygenator device of FIG. 2, according to embodiments of the present disclosure,

FIG. 7 is an exploded view of a plurality of hollow fiber mat layers prior to stacking them in a potting mold and resulting in the potted body of FIG. 6, according to embodiments of the present disclosure,

FIG. 8 illustrates a first element of the plurality of hollow fiber mat layers of FIG. 7, according to embodiments of the present disclosure,

FIG. 9 illustrates a second element of the plurality of hollow fiber mat layers of FIG. 7, according to embodiments of the present disclosure, and

FIG. 10 illustrates one of the plurality of hollow fiber mat layers of FIG. 7, prior to being cut, according to embodiments of the present disclosure.

### DETAILED DESCRIPTION

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

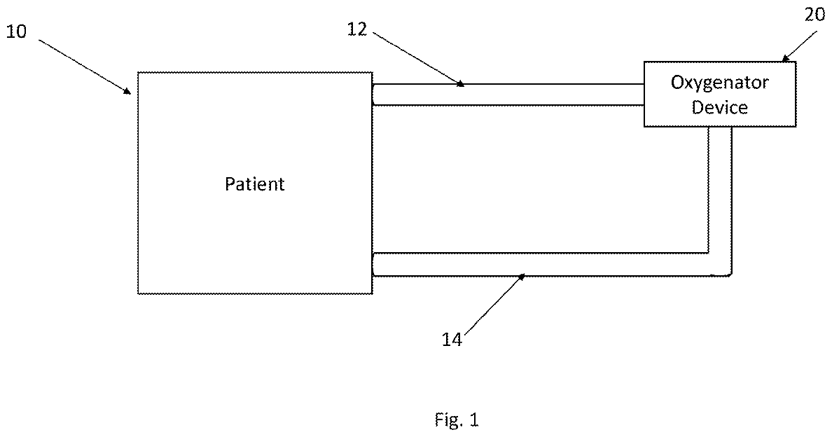

FIG. 1 is a schematic view of system comprising a patient 10 requiring extracorporeal blood circulation. In these embodiments, the patient 10 is connected through a first tubing 12 to an extracorporeal blood circulation device 20, also referred to generally as an oxygenator device 20 throughout, such that blood can be transferred from the patient 10 through the first tubing 12 to the oxygenator device 20. The oxygenator device 20 includes a second tubing 14 that extends from the oxygenator device 20 to the patient 10 for transferring blood that has been circulated within the device 20 back to the patient 10. In these embodiments, O₂ and CO₂ are exchanged between the blood and a gas mixture within the oxygenator device 20, as will be described further herein. The oxygenator device 20 is also configured for exchanging temperature (hot or cool temperatures) between the blood and heating/cooling (H/C) fluid within the oxygenator device 20. In some embodiments, the H/C fluid is water or a water solution.

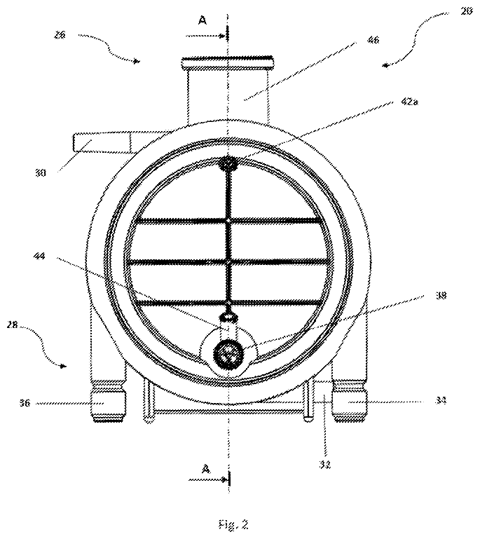

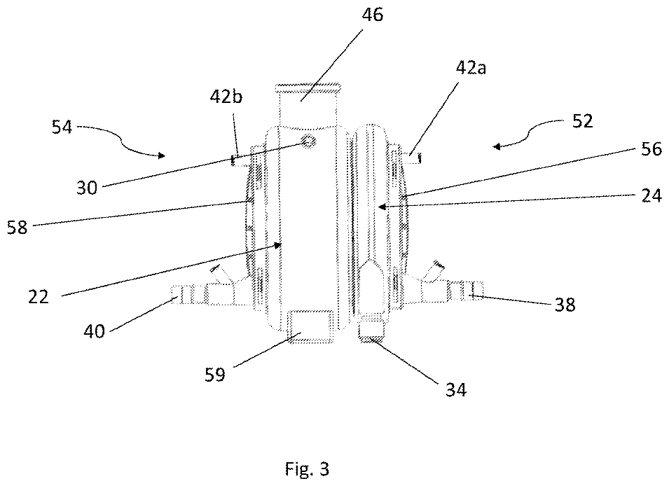

FIG. 2 is a front view of an oxygenator device 20 having an oxygenator module (not shown) and a heat exchanger module (not shown). The oxygenator device 20 has an upper portion 26 and a lower portion 28. In various embodiments, the oxygenator 20 includes a gas inlet port 30 configured for receiving a gas mixture, a gas outlet port 32 configured for exporting a gas mixture, a H/C fluid inlet port 34 for receiving H/C fluid, an H/C fluid outlet port 36 for exporting H/C fluid, a blood inlet port 38 for receiving blood from the patient 10 (FIG. 1), and a blood outlet port (not shown) for exporting blood from the oxygenator device 20 back to the patient 10. The oxygenator device 20 further includes a venous sampling port 44 and a first purging port 42a, as will be described further with reference to FIG. 3. In some embodiments, the oxygenator device 20 additionally includes a bracket attachment 46 to allow for attachment and rotation of the oxygenator device 20.

FIG. 3 is a side view of the oxygenator device 20. As illustrated, the oxygenator device 20 includes a front portion 52 and a rear portion 54. The front portion 52 includes a blood inlet end cap 56 having the blood inlet port 38 configured for receiving blood from first tubing 12 (FIG. 1) and the rear portion 54 includes a blood outlet end cap 58 having the blood outlet port 40 for providing an exit for the blood to return to the patient 10 (FIG. 1) through the second tubing 14 (FIG. 1). Additionally, as illustrated in FIG. 3, the oxygenator device 20 includes the gas inlet port 30 on the side surface of the oxygenator device 20. The oxygenator device 20 additionally includes a plurality of purging ports 42 including at least the first purging port 42a and a second purging port 42b. Purging ports 42a, 42b may allow for removal of air during an initial priming phase of the oxygenator device 20 prior to use with the patient. During operation, i.e., when blood and gas flow through the device 20, the purging ports 42a, 42b may be reopened for removing entrapped air from blood. Additionally, the purging ports 42a, 42b may be opening after operation of the device 20, i.e., when blood is no longer flowing through the device 20, to ensure proper emptying of any blood from the device 20 and returning it to the patient. The oxygenator device 20 also includes a pedestal 59 positioned on a bottom surface of the device 20 for supporting and stabilizing the device 20.

As previously described with reference to FIG. 2, the oxygenator device 20 includes the front portion 52 and the rear portion 54. In these embodiments and as illustrated in FIG. 3, the front portion 52 encompasses at least a portion of the heat exchanger module 24 and the rear portion 54 encompasses at least a portion of the oxygenator module 22.

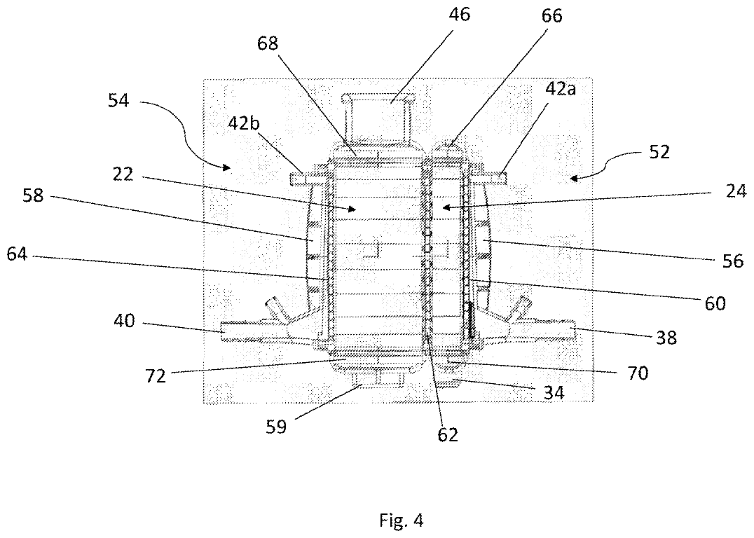

FIG. 4 illustrates a lateral cross section of the oxygenator device 20 taken along line A-A of FIG. 2. In the illustrative embodiment of FIG. 4, the oxygenator device 20 includes the oxygenator module 22 and the heat exchanger module 24 positioned adjacent the oxygenator module 22. Both modules 22, 24 are provided with hollow fiber mat layers vertically stacked one adjacent to the other. The heat exchanger module 24 is bordered on a right side, towards the front portion 52 of the oxygenator device 20, by a blood inlet distribution grid 60. The blood inlet distribution grid 60 receives the inputted blood from the blood inlet port 38 and distributes it within the blood inlet distribution grid 60 before flowing into the heat exchanger module 24. Heat exchanger module 24 is bordered on the left side, towards the rear portion 54, by a separation grid 62 which provides a physical separation between the oxygenator module 22 and the heat exchanger module 24 and distributes blood flowing past the heat exchanger module 24 towards the oxygenator module 22. The heat exchanger module 24 is thus on the opposing side of separation grid 62 relative to the oxygenator module 22 which is positioned further towards the rear portion 54 of the oxygenator device 20. The heat exchanger module 24 is positioned vertically below an H/C fluid inlet chamber 66 and positioned vertically above the pedestal 59 and an H/C fluid outlet chamber 70. The oxygenator module 22 is also bordered on the left side, towards rear portion 54 of the oxygenator device 20, by a blood outlet collection grid 64 which is configured for collecting the blood flowing from the oxygenator module 22 before it exits through the blood outlet port 40. As illustrated, vertically positioned above the oxygenator module 22 is a gas inlet chamber 68 and the bracket attachment 46. Vertically positioned below the oxygenator module 22 is the gas outlet chamber 72.

As such, each of the oxygenator module 22 and the heat exchanger module 24 generally include two portions, or two halves. As will be further described below, the oxygenator module 22 has a portion configured for communication with the gas inlet chamber 68 and a portion configured for communication with the gas outlet chamber 72. Similarly, the heat exchanger module 24 has a portion configured for communication with the H/C fluid inlet chamber 66 and a portion configured for communication with the H/C fluid outlet chamber 70. As illustrated, the front portion 52 of the oxygenator device 20 includes the blood inlet port 38 and the rear portion 54 of the oxygenator device 20 includes the blood outlet port 40. As a result of this configuration, when the blood is driven to pass through both the heat exchanger module 24 and the oxygenator module 22 in order to flow from the blood inlet port 38 to the blood outlet port 40, the blood is able to come into contact (by interposition of the appropriate hollow fiber membranes in the heat exchanger module 24 and the oxygenator module 22), with the fluid mixtures and the gas for sufficient heat and gas exchange.

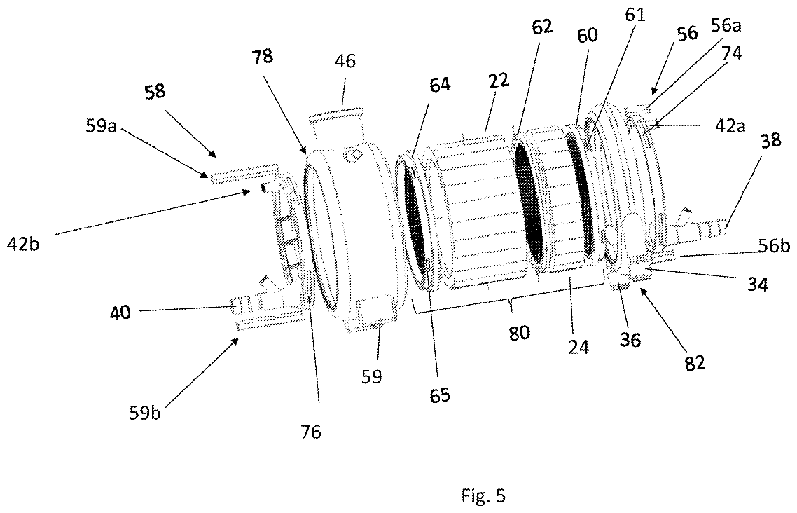

FIG. 5 is an exploded view of the oxygenator device 20 illustrating the component assembly within the oxygenator device 20. Referring to it, the oxygenator device 20 includes a blood path comprising the blood inlet end cap 56 with the blood inlet port 38 and the purging port 42a, the potted body 80, and the blood outlet end cap 58 with the blood outlet port 40 and the purging port 42b. The potted body 80 includes, embedded all together in one (potted) piece only, the blood inlet distribution grid 60, the heat exchanger module 24, the separation grid 62, the oxygenator module 22, and the blood outlet collection grid 64. Access for H/C fluid and gas to the inner lumens of heat exchanger 24 and oxygenator 22 hollow fibers is made possible through the hollow fiber open ends on the potted body outer surface.

The blood inlet end cap 56 is provided with a plurality of peripheral cavities 74 that mechanically fit into the corresponding peripheral notches 61 on the blood inlet distribution grid 60 of the potted body 80. Air tightness between blood inlet end cap 56 and blood inlet distribution grid 60 is obtained by resin casting along the two circular contact surfaces of the blood inlet end cap 56 and the blood inlet distribution grid 60. Similarly, on the opposite end of the potted body 80, the blood outlet end cap 58 includes a plurality of peripheral cavities 76 that mechanically fit into the corresponding peripheral notches 65 on the outlet collection grid 64 of the potted body 80. Air tightness between blood outlet end cap 58 and outlet collection grid 64 is obtained by resin casting along the circular contact surfaces of the blood outlet end cap 58 and outlet collection grid 64. In this way, the entire blood compartment, including blood inlet end cap 56, blood outlet end cap 58, and potted body 80, is joined in one air tightened piece.

Blood enters the oxygenator device 20 through blood inlet port 38 of blood inlet end cap 56, crosses blood inlet distribution grid 60, the stacked hollow fiber mat layers forming the heat exchanger module 24, the separation grid 62, the stacked hollow fiber mat layers forming the oxygenator 22, the outlet collection grid 64 and reaches the blood outlet port 40 of blood outlet end cap 58. The blood inlet distribution grid 60, the separation grid 62, and the outlet collection grid 64 are circular plastic parts with relatively large bores (from 1 to 8 mm) throughout their surfaces and are configured for keeping the elements of the heat exchanger module 24 and the oxygenator module 22 in place and assuring an even distribution of blood flowing across them. The remaining parts of the device 20 comprise external housings enclosing the H/C fluid compartment and the gas compartment, i.e., the H/C fluid collector 82 (including the H/C fluid inlet port 34 and the H/C fluid outlet port 36) and the gas collector 78 (including the gas inlet port 30 and the gas outlet port 32, as shown in FIG. 2, and including also the bracket attachment 46 and the pedestal 59).

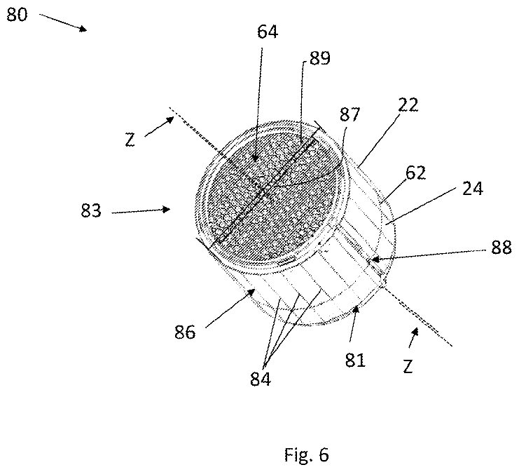

The H/C fluid collector 82 is positioned externally over the potted body 80 portion corresponding to the heat exchanger module 24 and assembled to the blood inlet end cap 56 by means of resin casting to air tighten the circular right side of the H/C fluid compartment. The left circular edge of the H/C fluid collector 82 is positioned externally in correspondence of the separation grid 62 and is air tightened to the potting body 80 by resin casting. In this way, the entire H/C fluid compartment is air tightened. The H/C fluid compartment is divided into two halves including an H/C fluid inlet chamber 66 and an H/C fluid outlet chamber 70 (shown in FIG. 4) by means of two sealing gaskets 56a, 56b which are inserted along two alignment features, illustratively two diametrically opposed and longitudinal grooves 88, present on the outer surface of the potted body 80 (shown in FIG. 6). The H/C fluid flows inside the H/C fluid compartment to (and from) the heat exchanger module 24 through the gap between the inner surface of the H/C fluid collector 82 and the outer surface of the potted body 80, thus forming the H/C fluid inlet chamber 66 and the H/C fluid outlet chamber 70.

Similarly, the gas collector 78 is positioned externally in correspondence with the outer surface of the potted body 80 relative to the oxygenator module 22 and assembled with the blood outlet end cap 58 by means of resin casting in order to air tighten the left circular side of the gas compartment of the device 20. The right circular edge of the gas collector 78 is positioned externally next to the H/C fluid collector 82 in correspondence of the separation grid 62 and is air tightened to the potting body 80 by resin casting. In this way, the gas compartment is entirely air tightened. Also, the gas compartment is divided in two halves: a gas inlet chamber 68 and a gas outlet chamber 72 (shown in FIG. 4) by means of two sealing gaskets 59a, 59b which are inserted along two alignment features, illustratively two diametrically opposed and longitudinal grooves 88 provided on the outer surface of the potted body 80 (as per FIG. 6). The gas flows to (and from) the oxygenator module 22 through the gas compartment given by the gap between the inner surface of gas collector 78 and outer surface of the potted body 80, forming the gas inlet chamber 68 and the gas outlet chamber 72.

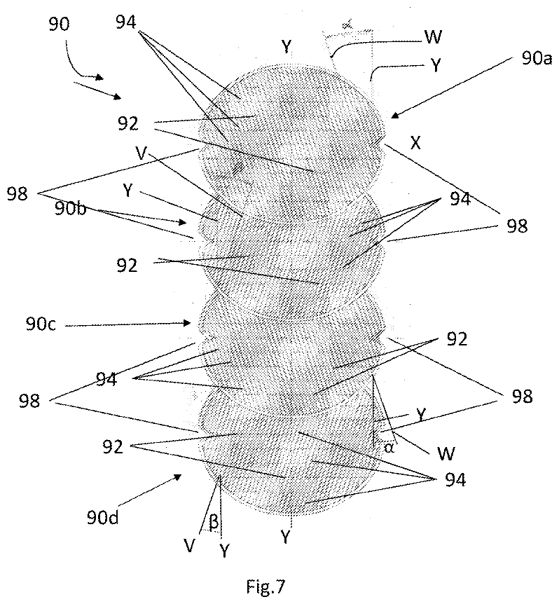

FIG. 6 is a perspective view of the potted body 80 of the oxygenator device 20 after having been demolded from the potting mold and having faceted a lateral outer surface 86 of the potted body 80, which provides it with a multifaceted polynomial shape extending along the axis Z and opens the ends of the hollow fibers of both 22 and 24 modules, thus giving access to their internal lumens for H/C fluid and gas circulation. Lines 84 indicate some of the many faces obtained by faceting. The potted body 80 is formed by centrifugation at high speed around the axis Z of the cylindrical potting mold, pre-filled with a number of hollow fiber mat layers 90 (shown in FIG. 7) stacked on top of one another to compose both the heat exchanger module 24 and the oxygenator module 22. A certain amount of resin, which may usually be polyurethane, initially liquid, is injected into the rotating mold and solidifies against the wall of the mold, pressed by centrifugal force. The difference between outer potting diameter 89 and inner potting diameter 87 is usually in the range of 7 to 10 mm (preferably 8 mm). Outer potting diameter 89 may be between 7 to 18 cm, and in preferred embodiments, is 12 cm. Therefore, the potted body 80 offers a large and unobstructed internal passage to the blood flow providing the benefit of substantially reduced blood pressure drop values for the oxygenator device 20. The potted body 80 includes a rear end 81 and a front end 83. The hollow fiber mat layers of the heat exchanger module 24 are positioned towards the rear end 81 and stacked adjacent the hollow fiber mat layers of the oxygenator module 22 which is positioned towards the front end 83. The separation grid 62 is interposed between the heat exchanger module 24 and the oxygenator module 22.

The potted body 80 has an outer surface or periphery 86, which in various embodiments, is provided with alignment features concurring with alignment features of the plurality of hollow fiber mat layers 90 to confer an aligned orientation to the entire oxygenator device 20, as will be further described. In embodiments, the alignment features of the potted body 80 include at least one groove extending longitudinally along the outer surface 86 of the potted body 80. For example, in the embodiment of FIG. 6, the alignment features comprise two longitudinal grooves 88 diametrically opposed parallel to the axis Z. The longitudinal grooves 88 may have a depth of 3 to 6 mm. In preferred embodiments, the depth of the longitudinal grooves 88 may be 4 mm. FIG. 6 shows the potted body 80 after faceting (slicing) its outer periphery 86, which, as explained, results in opening the extremities of the hollow fibers of the plurality of potted and stacked hollow fiber mat layers 90 (as per FIG. 7). Slices must have a depth sufficient for removing an outer layer of polyurethane and exposing the open lumens of each of the plurality of hollow fibers of the plurality of hollow fiber mat layers so to allow the circulation of fluids (gas mixture and the H/C fluid) inside the hollow fiber lumens. Blood flows around (i.e., outside of) the hollow fibers. Slicing depth may range from 1 mm to 4 mm. In preferred embodiments, slicing depth may be approximately 2 mm.

In various embodiments, the longitudinal grooves 88 of the potted body 80 create two general halves of the potted body 80. In such embodiments, after slicing the outer periphery 86 of the potted body 80, the open ends of each of the plurality of hollow fibers of the plurality of hollow fiber mat layers of the oxygenator device 20 are placed in communication with various of the oxygenator device 20 chambers, as previously disclosed. For example, a first half of the oxygenator module 22 comprises open hollow fiber terminations that are in communication with the gas inlet chamber 68 while the open hollow fiber terminations at their opposed ends (i.e., a second half of the hollow fiber terminations) are in communication with the gas outlet chamber 72. The plurality of hollow fibers of the plurality of hollow fiber mat layers of the heat exchanger module 24 are organized in a similar manner, with a first half of the hollow fiber open terminations enclosed within the H/C fluid outlet chamber 70, and their opposed open ends enclosed within the H/C fluid inlet chamber 66. As disclosed above, these four chambers are separated and sealed from each other by means of the two sealing gaskets 56a, 56b and the two sealing gaskets 59a, 59b (FIG. 5) and are located inside the gas collector 78 and the H/C fluid collector 82 and inserted along the grooves 88. Appropriate sealing means among the four chambers may be obtained not only as described above by gaskets, but also, for example by sealing them with resin (epoxy or others) or any other suitable sealing material.

The potted body 80 is made by stacking its various elements (i.e., the grids and the hollow fiber mat layers of both the oxygenator module 22 and the heat exchanger module 24) directly into a cylindrical potting mold according to a certain sequence and orientation. As shown in FIG. 7-10, the elements to be stacked, referenced 90, are flat circular layers with outer diameter equal to the mold inner diameter. Each of them has two diametrically opposed grooves 98 to be introduced into two corresponding protrusions of the potting mold. The layer of the blood inlet distribution grid 60 is first to be introduced into the mold, followed by a stack of the plurality of hollow fiber mat layers 90 for the heat exchanger module 24. Next, the layer of the separation grid 62 is inserted into the mold, followed by a stack of the plurality of hollow fiber mat layers 90 for the oxygenator module 22, and lastly the layer of the blood outlet collection grid 64 is inserted. The number of the plurality of stacked hollow fiber mat layers 90 depends on the size of the oxygenator device 20 to be realized. At this point, the loaded potting mold undergoes one step centrifugation with polyurethane resin about the longitudinal axis Z. After centrifugation, the cylindrical potted body 80 is demolded, sliced (i.e., faceted on its lateral outer surface) and assembled with the other parts of the oxygenator device 20 as already described.

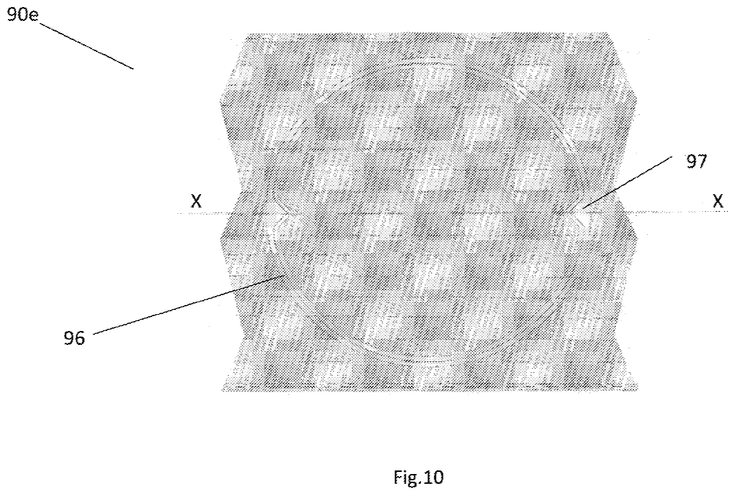

The plurality of hollow fiber mat layers 90 stacked into the potted body 80 are described in further detail with reference to FIGS. 7-10. FIG. 7 illustrates an exploded view of some layers of the plurality of hollow fiber (HF) mat layers 90 as they are piled into the potting mold. The plurality of hollow fiber mat layers 90 are circularly shaped, each including a plurality of threads 92, also referred to as warp threads, and a plurality of hollow fibers (HFs) 94, parallel one another. Such mat layers are obtained by hot sealing and cutting their circular outer periphery as will be described further with reference to FIG. 10.

In various embodiments, hollow fibers 94 are microporous hydrophobic membranes made of materials such as, but not limited to, polypropylene or polymethyl pentene in the oxygenator module 22 and non-microporous membranes made of polyethylene, or polyurethane, in the heat exchanger module 24. The plurality of microporous hollow fibers 94 of the oxygenator module 22 are configured such that they allow gases to diffuse through the porosity of hollow fibers 94 into blood (O₂) and vice versa (CO₂). In the heat exchanger module 24, the plurality of hollow fibers 94 permits only heat transfer between blood and H/C fluid (or vice versa). The plurality of threads 92 may be made of polyester fibers.

As illustrated in FIG. 7-9, each hollow fiber mat layer 90 includes a plurality of parallel threads, all oriented in the direction of the axis X and comprises alignment features resulting in the alignment features of the potted body 80 (used to orient the chambers of the H/C fluid and gas collectors 82 and 78). In the illustrative embodiment of FIGS. 7-9, the alignment features are provided by at least two grooves 98 on either side of each mat layer 90, relatively aligned with one another and diametrically opposed to define the lateral axis X. In this way, the alignment features of each mat layer 90 are aligned in a direction parallel with the direction of the plurality of threads 92. The particular position and orientation of the two grooves 98 are such that the plurality of hollow fibers 94 in each layer are divided into two halves (upper and lower), with every single fiber having one termination in the upper half and the other in the lower half No fibers are “short-circuited”, i.e., have both terminations in the same half and so are fully exploited in the exchange process. A fluid (either H/C fluid, or gas) entering the fiber lumen at one end, flows along the entire fiber and exits at the opposite end, as the grooves 98, once the layers are potted, generate the grooves 88 of the body 80, which also align the inlet and outlet chambers of the device 20.

Another particularity shown in FIGS. 7-9 is that while the plurality of threads 92 of each hollow fiber mat layer 90 extends along the direction of the lateral axis X, indicating the orientation of each of the plurality of hollow fiber mat layers 90, the plurality of hollow fibers 94 of each of the plurality of hollow fiber mat layers 90 are distorted at an angle with reference to the direction of an axis Y, wherein axis Y is orthogonal to axis X. Therefore, the plurality of hollow fibers 94 are positioned on each of the plurality of hollow fiber mat layers 90 with threads 92 parallel to axis X, but distorted relative to the axis Y such that they are generally angled with reference to the direction of axis Y. In these embodiments, as shown in FIG. 7, the directionality of the plurality of hollow fibers 94 in comparison to the hollow fiber mat layers 90 positioned adjacent one another have alternating distortion angles, while the orientation of the plurality of warp threads 92 remains consistent and parallel to axis X. This allows the criss-crossing orientation of the hollow fibers 94, which is essential in maximizing the heat and gas exchange performances of the device 20, while the plurality of mat layers 90 is oriented in the direction of the X axis.

For example, in FIG. 7, a first hollow fiber mat layer 90a of the plurality of hollow fiber mat layers 90 includes the plurality of threads 92 extending parallel along the axis X and the plurality of hollow fibers 94 extending in a direction that is parallel with an axis W, which extends at a distortion angle α relative to axis Y where a may be higher than 0 up to 25 degrees. In preferred embodiments, the angle α may range from a value of 12.5 degrees to 25 degrees. Further, a second hollow fiber mat 90b of the plurality of hollow fiber mat layers 90 is placed below the first hollow fiber mat layer 90a and illustrated having a plurality of threads 92 extending along lateral axis X and a plurality of hollow fibers 94 extending in a direction that is generally parallel to axis V. In these embodiments, axis V is angled relative to axis Y by an angle β. In embodiments, the angle α and the angle β are disposed on opposite sides of the axis Y. In various embodiments, the value of angle β may be higher than 0 up to 25 degrees. In preferred embodiments, the angle β may range from 12.5 degrees to 25 degrees. In further preferred embodiments, the value of angle β may be equal and opposite in direction to the angle α. For example, angle α may have a value of 10 degrees while angle β may have a value of −10 degrees (i.e., in the opposite direction). In such case, the plurality of hollow fibers 94 of the respective adjacent first hollow fiber mat layer 90a and the second hollow fiber mat layer 90b are offset by an angle of approximately double the value of α, or 2xα (i.e., 20 degrees). In further embodiments, the value of angle β may be different than the value of angle α. For example, in some embodiments, angle α may have a value of 10 degrees while angle β may have a value of −15 degrees. Further, while angle α and angle β are illustrated as being substantially the same among the various layers of the plurality of hollow fiber mat layers 90, their values may be different among layers of the plurality of hollow fiber mat layers 90.

Angling the hollow fibers 94 of the respective adjacent first and second hollow fiber mat layers 90a, 90b in opposite directions (as it is for W and V in FIG. 7) versus direction Y allows to increase the offset between hollow fibers 94 of adjacent hollow fiber mat layers 90. Angling among the plurality of hollow fibers 94 in the stacked hollow fiber mat layers 90 is essential for providing optimized mass transfer between gases and H/C fluid and blood flowing through the oxygenator device 20.

A third hollow fiber mat layer 90c of the plurality of hollow fiber mat layers 90 is placed below the second hollow fiber mat layer 90b. In the illustrated embodiment of FIG. 7, the third hollow fiber mat layer 90c is identical to the first hollow fiber mat layer 90a such that it has a plurality of threads 92 extending along lateral axis X, the plurality of hollow fibers 94 are aligned with axis W, and the angle between lateral axis Y and axis W is α. Further, a fourth hollow fiber mat layer 90d may be positioned beneath the third hollow fiber mat layer 90c. In the illustrative embodiment of FIG. 7, the fourth hollow fiber mat layer 90d is identical to the second hollow fiber mat 90b such that the plurality of threads 92 is parallel to lateral axis X, and the plurality of hollow fibers 94 is directed generally parallel with axis V (at an angle β vs Y). In general, angles α and β of 90c and 90d may be different in values from those of first hollow fiber mat layer 90a and the second hollow fiber mat layer 90b.

This alternating orientation among the plurality of hollow fiber mat layers 90 allows for the plurality of hollow fibers 94 in each of the plurality of hollow fiber mat layers 90 to be oriented at an angle that is generally offset from the direction of the plurality of hollow fibers 94 of the hollow fiber mat layers 90 positioned adjacent, while the orientation of the threads 92 in all stacked hollow fiber mat layers 90 is substantially kept parallel throughout the device. In the plurality of hollow fiber mat layers 90, the hollow fiber mat layers 90a-d may include one or a group of more layers (up to 10) of hollow fiber mat layers 90, each group of layers being characterized by hollow fibers 94 distorted at different angles compared to the axis Y orthogonal to the direction of all the threads 92 (all of them parallel).

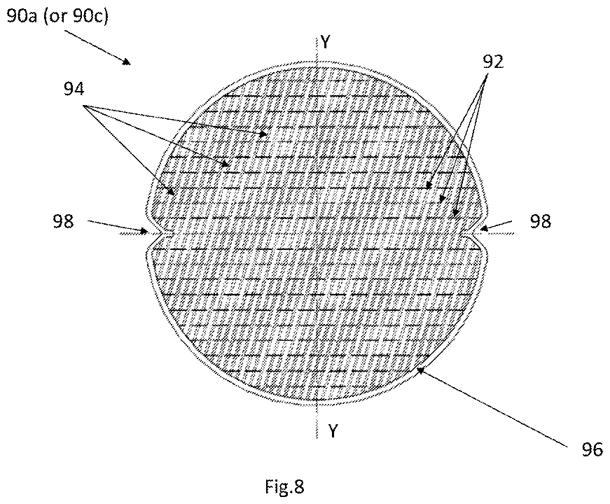

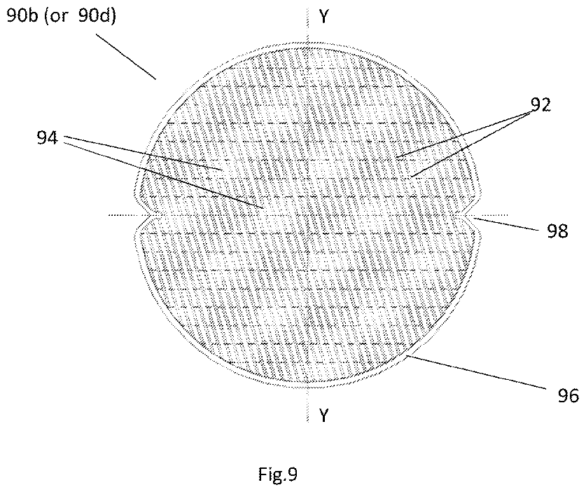

FIG. 8 is a frontal view of the first (or third) hollow fiber mat layer 90a (or 90c) of FIG. 7 and FIG. 9 is a frontal view of the second (or fourth) hollow fiber mat layer 90b (or 90d) of FIG. 7. FIGS. 8 and 9 show equal distortion angles in opposite directions for the hollow fibers 94 of the first and second hollow fiber mat layers 90a, 90b and equal orientation of the threads 92. Each of the plurality of hollow fiber mat layers 90 is obtained by circularly hot sealing, cutting the hollow fiber mat layer 90 away and separating the circular HF mat layer 90 from the hollow fiber mat layer 90 material outside of the hot sealed area. The outer periphery 96 includes the two diametrically opposed grooves 98 parallel to the threads 92, which, as explained, allow the correct orientation and alignment of the layers in the potting mold and subsequently generate the longitudinal grooves 88 in the potted body. The hot sealing may be accomplished through use of a conventional hot sealing machine or other appropriate methods of hot sealing.

FIG. 10 shows a hot sealed hollow fiber mat layer 90e, prior to being cut away and separated from the other mat material, i.e., before reaching the stage shown in FIGS. 8 and 9. The hot sealed crown area 97 is twice as large as the outer periphery 96, which is the only one remaining in place after cutting. Typically, hot sealed crown area 97 is 2 to 4 mm thick, preferentially 3 mm. Cutting may be done through various means, such as slicing, punch cutting, or any other suitable means. The process may be done on individual mat layers or collectively on as many individual (or group of) hollow fiber mat layers 90 as may be desired. Then, the hollow fiber mat layers 90 can be stacked relative to one another as shown in FIG. 7 into the potting mold. Before hot sealing the layer, the hollow fiber mat material must be correctly positioned by having threads 92 parallel to the X axis and hollow fibers 94 distorted of an angle α, or β, compared to the direction Y (orthogonal to X). Besides, the hot sealed grooves 98 must be diametrically opposed to one another and oriented parallel to the X axis. The use of hot sealing for getting the layers is essential in that it maintains the relative positionings among the plurality of hollow fibers 94 and plurality of threads 92 (i.e., creating the distortion angles) and marks the correct position of the two grooves 98, by freezing them in place and making it possible a subsequent stacking process where all the hollow fiber mat layers 90 are aligned with the plurality of threads 92 parallel to a first direction and the plurality of hollow fibers 94 in adjacent layers alternatively distorted compared to the direction orthogonal to the first direction. In conclusion, the stacking process can be done without the need for any rotation of the plurality of hollow fiber mat layers 90, because the angling among the hollow fibers 94 in the different hollow fiber mat layers 90, given by the particular formation process of each layers 90 described above, provides sufficient criss-crossing orientation among the hollow fibers. This is key in improving heat and gas transfer efficiency, at the same time, increasing the ease of assembly of the oxygenator device 20 (which may be easily automated) and decreasing the chances of inconsistency during manufacturing.

The oxygenator module 22 has been illustrated so far in embodiments as stacked adjacent to the heat exchanger module 24 without any angle of rotation relative to one another. Various other embodiments are possible, such as wherein the oxygenator module 22 and the heat exchanger module 24 are stacked adjacent one another but rotated at an angle around the Z axis. The angle may have a value between zero (as shown in FIGS. 2-6) and 90 degrees. In such embodiments, the potted body 80 is molded in two steps to accommodate the offset positioning of the heat exchanger module 24 and the oxygenator module 22, no longer being stacked parallel to one another. Further, the orientations of the H/C fluid inlet chamber 66 and H/C fluid outlet chamber 70 and the gas inlet chamber 68 and the gas outlet chamber 72 would correspondingly be rotated in order to maintain their communication with the respective halves of the hollow fiber terminations within the oxygenator module 22 and the heat exchanger module 24. Moreover, further embodiments are possible where the heat exchanger module 24 is missing and only the oxygenator module 22 is included in the device 20.

Numerous characteristics and advantages have been set forth in the preceding description, including various alternatives together with details of the structure and function of the devices and/or methods. Moreover, the inventive scope of the various concepts addressed in this disclosure has been described both generically and with regard to specific examples. The disclosure is intended as illustrative only and as such is not intended to be exhaustive. For example, the various embodiments of the present disclosure are described in the context of medical applications but can also be useful in non-medical applications. It will be evident to those skilled in the art that various modifications may be made, especially in matters of structure, materials, elements, components, shape, size, and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

1. An oxygenation device for use in connection with extracorporeal blood circulation, the device comprising:
a potted body including a first alignment feature defining an alignment axis;
an oxygenator module comprising a plurality of circular hollow fiber mat layers including a second alignment feature configured to align with the first alignment feature of the potted body, such that each of the plurality of hollow fiber mat layers is oriented with respect to the alignment axis;
a heat exchanger module comprising at least two of the plurality of circular hollow fibers mat layers;
a blood inlet port positioned adjacent a first end of the potted body;
a blood outlet port positioned adjacent a second end of the potted body that is opposite the first end;
a gas inlet port and a gas outlet port configured for guiding a gas mixture through a plurality of hollow fibers of the plurality of hollow fiber mat layers; and
a fluid inlet port and a fluid outlet port configured for providing and removing H/C fluid through a portion of the potted body;
wherein a first layer of the plurality of hollow fiber mat layers comprises a first plurality of parallel hollow fibers disposed at a first angle relative to a direction orthogonal to the alignment axis and a second layer of the plurality of hollow fiber mat layers includes a second plurality of parallel hollow fibers disposed at a second angle relative to a direction orthogonal to the alignment axis, the first and second angles disposed on opposite sides of a direction orthogonal to the alignment axis.

2. The device of claim 1, wherein the gas mixture is configured to pass through the at least two of the plurality of hollow fiber mat layers of the oxygenator module.

3. The device of claim 1, wherein the H/C fluid is configured to pass through the at least two of the plurality of hollow fiber mat layers of the heat exchanger module.

4. The device of claim 1, wherein the potted body further comprises a distribution grid positioned between the blood inlet port and the heat exchanger module.

5. The device of claim 1, wherein the potted body further comprises a separation grid positioned between the heat exchanger module and the oxygenator module.

6. The device of claim 1, wherein the potted body further comprises a collection grid positioned between the oxygenator module and the blood outlet port.

7. The device of claim 1, further comprising a first housing portion placed adjacent the first end of the potted body and a second housing portion positioned adjacent the second end of the potted body.

8. The device of claim 1, wherein the plurality of hollow fiber mat layers each comprise a plurality of threads coupled to the plurality of hollow fibers.

9. The device of claim 8, wherein the plurality of threads are aligned at an angle that is not orthogonal to the alignment of the plurality of hollow fibers.

10. An assembly of a plurality of hollow fiber mat layers for use in an extracorporeal blood circulation device, the assembly comprising:
a first hollow fiber mat layer and a second hollow fiber mat layer, positioned directly adjacent one another, each of the first and second hollow fiber mat layers comprising a plurality of parallel threads and a plurality of parallel hollow fibers;
wherein the plurality of threads of the first and second hollow fiber mat layers are positioned parallel to an alignment axis; and
wherein the plurality of hollow fibers of the first hollow fiber mat layer are disposed at a first angle with respect to a direction orthogonal to the alignment axis and the plurality of hollow fibers of the second hollow fiber mat layer are disposed at a second angle with respect to a direction orthogonal to the alignment axis, which defines a third axis on an opposite side of the direction orthogonal to the alignment axis as the first angle.

11. The assembly of claim 10, wherein the first and second hollow fiber mat layers are circular in shape.

12. The assembly of claim 10, wherein the second axis is angled relative to the third axis by between 0 and 50 degrees.

13. The assembly of claim 12, wherein each of the plurality of hollow fiber mat layers comprises at least two grooves that align with the at least two grooves of the hollow fiber mat layers positioned adjacent one another.

14. The assembly of claim 13, wherein each of the plurality of hollow fiber mat layers are aligned to have the same orientation as one another.

15. The assembly of claim 12, wherein the plurality of hollow fiber mat layer comprises a third hollow fiber mat layer positioned directly below the second hollow fiber mat layer, and a fourth hollow fiber mat layer positioned directly below the third hollow fiber mat layer.

16. The assembly of claim 15, wherein a plurality of hollow fibers of the third hollow fiber mat layer are aligned along the second axis and a plurality of hollow fibers of the fourth hollow fiber mat layer are aligned with the third axis.

17. An oxygenation device for use in connection with extracorporeal blood circulation, the device comprising:
a potted body including a blood inlet disposed near a first end and a blood outlet disposed near a second end opposing the first end, and an exterior lateral surface including a first alignment feature defining an alignment axis; and
an oxygenator module including a plurality of circular hollow fiber mat layers including a plurality of hollow fibers coupled to a plurality of threads, each of the layers including a second alignment feature configured to align with the first alignment feature of the potted body, such that each of the plurality of hollow fiber mat layers is oriented with respect to the alignment axis;
wherein a first layer of the plurality of hollow fiber mat layers includes a first plurality of parallel hollow fibers disposed at a first alignment angle relative to a direction orthogonal to the alignment axis and a second layer of the plurality of hollow fiber mat layers, disposed adjacent to the first layer, includes a second plurality of parallel hollow fibers disposed at a second alignment angle relative to a direction orthogonal to the alignment axis, the first and second angles disposed on opposite sides of the direction orthogonal to the alignment axis.

18. The oxygenation device of claim 17, wherein the first and second angles are opposite angles with respect to the alignment axis.

19. The oxygenation device of claim 17, wherein each of the first and second angles is between 0 and 25 degrees.

20. The oxygenation device of claim 19, wherein in each of the hollow fiber mat layers, the plurality of threads are disposed at an angle that is not orthogonal to the alignment of the plurality of hollow fibers.
